(19) United States
(12) Patent Application Publication
(10) Pub. No.: US 2014/0356940 A1
(43) Pub. Date: Dec. 4, 2014

(54) WASTE BIOFERMENTER, BASED ON TEMPERATURE CONTROL BY MEANS OF HEATING AND COOLING AN ADDED LIQUID OR GASEOUS SOLUTION FOR ENERGY-USE OPTIMIZATION

(71) Applicant: Marco Antonio Gonçalves De Mieres, Niteroi-RJ (BR)

(72) Inventor: Marco Antonio Gonçalves De Mieres, Niteroi-RJ (BR)

(73) Assignee: SOLI&MIÉRES ENERGIA SUSTENTABILIDADE E GERENCIAMENTE AMBIENTAL LTDA, Rio de Janeiro, RJ (BR)

(21) Appl. No.: 14/122,046

(22) PCT Filed: Aug. 8, 2012
(86) PCT No.: PCT/BR2012/000284

§ 371 Date: Mar. 26, 2014

(30) Foreign Application Priority Data
Aug. 8, 2011 (BR) PI1103837-3

(51) Int. Cl. C12M 1/34

(52) U.S. Cl. CPC C12M 41/12
USPC 435/303.1

(57) ABSTRACT

The present invention comprises a waste-bio-fermenter device based on temperature control by means of heating and cooling an added liquid or gaseous solution, for energy-use optimization. The device makes it possible to utilize, for energy purposes, biomass and gases that result from anaerobic biodigestion in the thermophilic phase, said device differing from biodigesters currently offered in the market in that its temperature control is monitored, resulting in a shorter fermentation time, better quality, for energy-use purposes, of the produced dry material, and a high yield. The waste bio-fermenter has an elongate form and is built in a metallic structure comprising double partition lateral walls, sloping bottom to allow the solution runoff, and a ceiling with a hatchway. In the space between the partition walls heating of the liquid or gaseous solution placed therein occurs and then the heat present in the solution is transmitted to the internal cell.

The present invention aims to provide a waste-bio-fermenter device based on temperature control by means of heating and cooling an added liquid or gaseous solution, for energy-use optimization.

The present stored waste bio-fermenter is an innovative product due to its conception. There are no bio-fermentation models currently available in the market similar to the proposed device, which has an elongate shape and is built as a metallic structure with double partition lateral walls, a sloping bottom to allow the solution runoff, and with a ceiling having a hatchway. In the space between the partition walls heating of the liquid or gaseous solution placed therein occurs and then the heat present in the solution is transmitted to the internal cell.

By means of the bio-fermentation technique in the thermophilic phase, with induced temperature control, the production of gas in the anaerobic phase helps in accelerating the decomposition of the organic matter stored in the bio-fermenter. The waste bio-fermenter provides a new way of storing waste matter, in a more convenient way and making better usage of space and time, allowing the device to be used in an optimal way, also allowing the energy use from the produced methane gas. The production of gas results from the way that the waste is treated, in which waste matter, iodine, and glycerol are mixed together at a controlled moisture content and thermophilic temperature.

The waste bio-fermenters' design and operation solve the drawbacks resulting from the irregular disposal of waste in cities. Once the waste bio-fermenters are grouped, they set up a sustainable alternative in the search for solutions for the environmental problems facing the planet.

The waste bio-fermenters' loading and placing operation, in the maturation position, is simple and practical, since the parts involved in the system are modules that fit onto each other, and can be displaced into any position.

There are various techniques and technologies available in the market. There are various types of bio-digesters, including Chinese, Indian, Navy, and the like. The techniques for using bio-digesters are batch or continuous flow processes. The waste bio-fermenter improves the biodigestion technique by reducing the fermentation time, producing a higher-quality dry matter, which contributes to mitigating the environmental problems caused by the irregular disposal of waste matter in cities.

The figures show the arrangement in which the waste bio-fermenter is built.

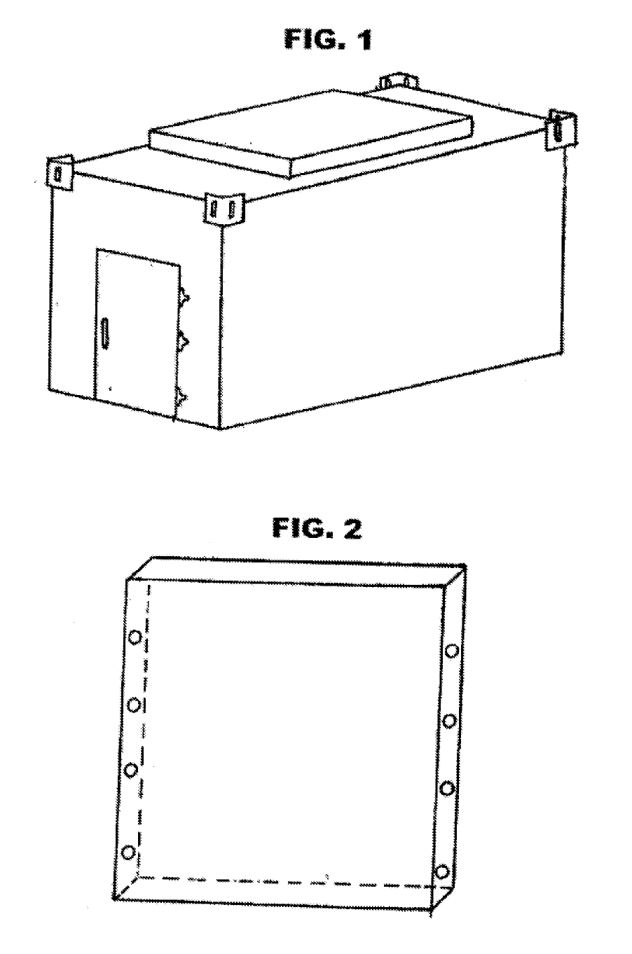

FIG. 1 depicts the module on its vertical form.

FIG. 2 depicts the double-sided lateral walls.

FIG. 3 depicts the ceiling with a hatchway and its fitting in detail for receiving another module.

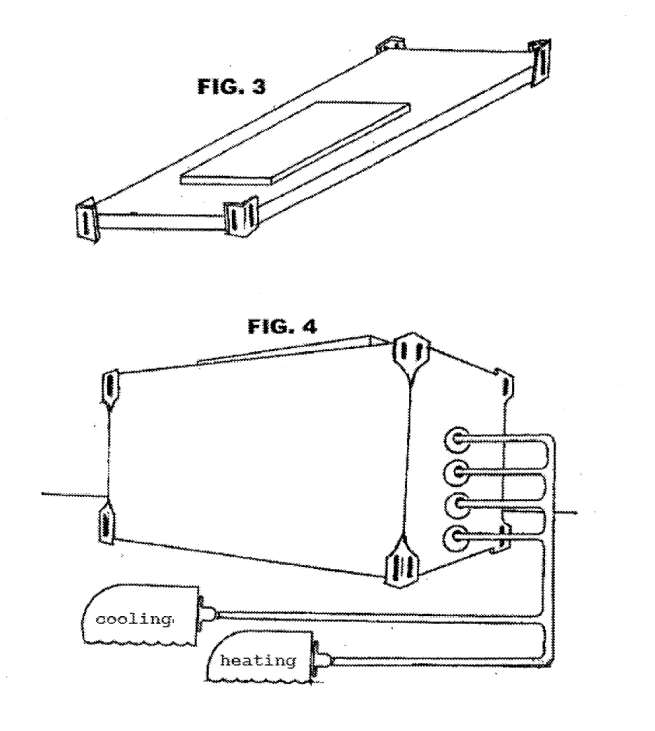

FIG. 4 shows the bottom of the module illustrating its inlet and outlet for cooling or heating, in detail.

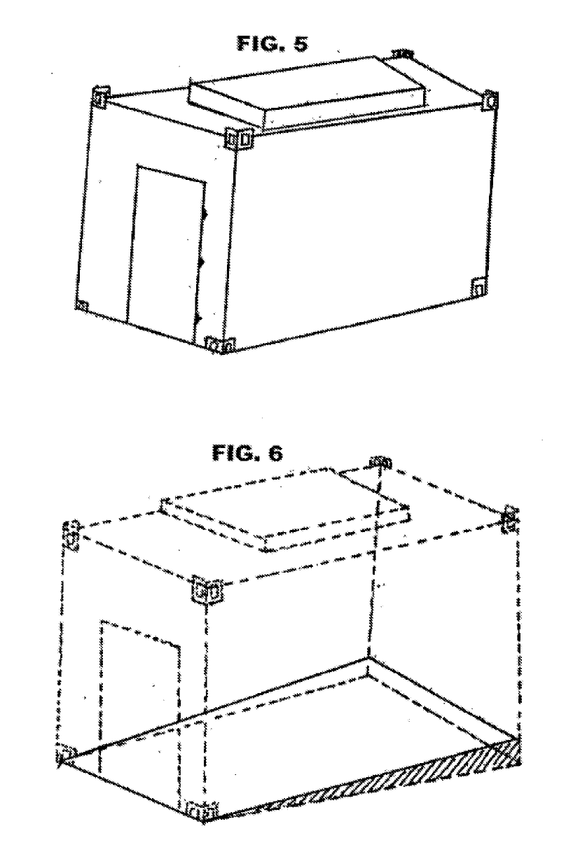

FIG. 5 shows the door for the access and maintenance of the equipment.

FIG. 6 depicts the sloping bottom.

1. Waste bio-fermenter, based on temperature control by means of heating and cooling an added liquid or gaseous solution for energy-use optimization,
characterized by comprising an elongate and vertical bio-fermenter (1), double-sided lateral walls and bottom (2), ceiling having a hatchway and a fitting slot for another module (3), inlet and outlet (4) for cooling and heating, an entrance door for the access and maintenance of instruments (5), sloping bottom (6),
wherein said waste bio-fermenter is an innovative model consisting of a better model for the storage of waste matter, thus being an environmentally-friendly way of disposing waste.
